(19) United States
(12) Patent Application Publication
(10) Pub. No.: US 2015/0342876 A1
(43) Pub. Date: Dec. 3, 2015

(54) METHODS AND PHARMACEUTICAL COMPOSITIONS FOR THE TREATMENT OF OCULAR INFLAMMATORY DISEASES

(71) Applicants: Francine BEHAR-COHEN, Paris (FR); Elodie BOUSQUET, Paris (FR)

(72) Inventors: Francine BEHAR-COHEN, Paris (FR); Elodie BOUSQUET, Paris (FR)

(73) Assignees: Institut National de la Santé et de la Recherche Médicale (INSERM), Paris (FR); UNIVERSITÉ PARIS DESCARTES, Paris Cedex 06 (FR)

(21) Appl. No.: 14/823,168

(22) Filed: Aug. 11, 2015

Related U.S. Application Data
(63) Continuation of application No. 13/978,091, filed on Oct. 9, 2013, now abandoned.

(30) Foreign Application Priority Data
Jan. 3, 2011 (EP) 11305007.4

(51) Int. Cl. A61K 9/00; A61K 31/573

(52) U.S. Cl. CPC A61K 9/0051; A61K 31/573; A61K 9/0048

(57) ABSTRACT

The current invention provides a new and original method for treatment of ocular inflammatory diseases. More particularly, the present invention relates a mineralocorticoid receptor agonist for use in the treatment of an ocular inflammatory disease.

### FIELD OF THE INVENTION

The present invention relates to methods and pharmaceutical compositions for the treatment of ocular inflammatory diseases.

### BACKGROUND OF THE INVENTION

Inflammation of the ocular tissues can occur by a variety of mechanisms and is associated, either primarily or secondarily, with a large number of disease conditions. For example, its aetiology may be infection, allergy, immunological reactions, or as a response to surgery, injury, or due to any other causes. Inflammation has also been strongly associated to retinal diseases such as Age related Macular degeneration (AMID) and diabetic retinopathy. The ocular inflammation causes pain, redness, irritation, watering, threatens visual function of the eye and may also change optical properties of the eye. Ocular inflammation, or intraocular inflammation, when unchecked or chronic can lead to permanent loss of vision. In fact, uveitis, or inflammation inside the eye, is the third leading cause of blindness in the United States, after diabetes and macular degeneration.

The treatment of ocular inflammation in general depends on the causative agents, the location and the severity of the inflammation. Infections represents less than a third of the cases and anti infective agents specific of the microbial causative agents most commonly require to be associated to anti inflammatory agents in order to preserve tissues integrity and transparency. Current treatments for inflammation of these tissues generally involve a part from systemic administration of antibiotics, glucocoticoids (systemic and loco regionally administered), and immune-system inhibitors, administered systemically. The difficulty of using these systemic drugs becomes apparent through damaging long-term side effects in the case of steroids, long-term drug resistance in the case of antibiotics, or insufficient long-term persistence at the target site in the case of signalling inhibitors. Moreover, the systemic inhibition of signalling within the immune system can have deleterious outcomes for individuals already afflicted with disease, whose susceptibility to additional complications is increased as a result of the systemic use of these treatments. Local treatments are therefore generally preferred when possible (only one eye affected, mild inflammation) but loco regional and local glucocorticoids are associated with frequent and severe side effects: glaucoma (about 50% of the case), cataract (80% of the cases) aggravation of infections (particularly herpes keratitis), delay of surface healing . . . .

For example, uveitis has been treated by various classes of compounds including steroids and nonsteroidal anti-inflammatory agents such as dexamethasone, flurometholone, prednisolone, indomethacin, aspirin, flubiprofen and diclofenac. However, a number of uveitic cases are not responsive to or become refractory to these drugs. Serious side effects including cataract, glaucoma, delayed wound healing, and altered prostaglandin production, and corneal complications including ulceration, perforation, and corneal and scleral melts have been reported with the use of topical steroids and nonsteroidal anti-inflammatory drugs. There is a need for novel anti inflammatory agents with reduced side effects.

Aldosterone, the endogenous ligand of the mineralocorticoid receptor (MR) in humans, is a steroid hormone that regulates salt and water homeostasis. Recently, additional pathophysiological effects in the renocardiovascular system have been identified. Besides genomic effects mediated by activated MR, rapid aldosterone actions that are independent of translation and transcription have been documented. For example, an additional pathophysiological mechanism described for aldosterone is the induction of inflammation which then leads to remodelling processes including fibrosis and cardiac hypertrophy. However involvement in aldosterone in ocular inflammation has not yet been investigated.

### SUMMARY OF THE INVENTION

The current invention provides a new and original method for treatment of ocular inflammatory diseases. More particularly, the present invention relates a mineralocorticoid receptor agonist for use in the treatment of an ocular inflammatory disease.

### DETAILED DESCRIPTION OF THE INVENTION

The present invention relates a mineralocorticoid receptor agonist for use in the treatment of an ocular inflammatory disease.

As used herein, the term “ocular inflammatory disease” refers to any ocular disease associated with inflammation and includes but is not limited to as conjunctivitis, keratitis, endothelitis, uveitis, choroiditis, retinitis, retinochoroiditis, anterior uveitis, intermediate uveitis, posterior uveitis, pan uveitis and inflammatory optic neuropathies.

As used herein, the term “mineralocorticoid receptor” or “MR” has its general meaning in the art and refers to the nuclear receptor subfamily 3, group C, member 2, (NR3C2) that is a receptor with high affinity for mineralocorticoids. The mineralocorticoid receptor is also called aldosterone receptor. The MR agonistic activity of a compound may be determined using various methods as described in J, Souque A, Wurtz J M, Moras D, Rafestin-Oblin M E. Mol Endocrinol. 2000 August; 14(8):1210-21; Fagart J, Seguin C, Pinon G M, Rafestin-Oblin M E. Mol Pharmacol. 2005 May; 67(5):1714-22 or Hellal-Levy C, Fagart J, Souque A, Wurtz J M, Moras D, Rafestin-Oblin M E. Mol Endocrinol. 2000 August; 14(8):1210-21. Typically, the transfection of the human mineralocorticoid receptor in COS cells together with a luciferase-expressing reporter gene allows to measure its transactivation activity in the presence of a candidate compound.

As used herein, the term “mineralocorticoid receptor (MR) agonist” is a natural or synthetic compound which binds the mineralocorticoid receptor to activate said mineralocorticoid receptor site for initiating a pathway signalling and further biological processes. According to the invention agonist include but are not limited to peptides, polypeptides, protein, nucleic acids such as aptamers, small organic molecules (natural or not).

According to the invention; the mineralocorticoid receptor (MR) agonist according to the invention is not a glucocorticoid.

As used herein the term “glucocorticoid” has it general meaning in the art and refers to compounds that bind and activate the glucocorticoid receptor (GR) also known as NR3C1 (nuclear receptor subfamily 3, group C, member 1).

In the context of the present invention, mineralocorticoid receptor agonists are preferably selective for the mineralocorticoid receptor as compared with the related receptors such as androgen receptor, estrogen receptors, glucocorticoid receptor, progesterone receptor, thyroid hormone receptors, peroxisome proliferator-activated receptors, retinoic acid receptor, farnesoid x receptor, pregnane x receptor, liver X receptor, vitamin D receptor, retinoid x receptor and the constitutive androstane receptor. By “selective” it is meant that the affinity of the antagonist for the mineralocorticoid receptor is at least 10-fold, preferably 25-fold, more preferably 100-fold, still preferably 500-fold higher than the affinity for the related receptors.

In one embodiment, the mineralocorticoid receptor agonist is a small organic molecule.

The term “small organic molecule” refers to a molecule of a size comparable to those organic molecules generally used in pharmaceuticals. The term excludes biological macromolecules (e. g., proteins, nucleic acids, etc.). Preferred small organic molecules range in size up to about 5000 Da, more preferably up to 2000 Da, and most preferably up to about 1000 Da.

In a particular embodiment, the MR agonist according to the invention is selected from the group consisting of aldosterone or aldosterone analogs.

As used herein, the term “aldosterone” refers to 11β,21-dihydroxy-3,20-dioxopregn-4-en-18-al that is the natural endogen agonist of mineralocorticoid receptor.

As used herein, the term “aldosterone analog” refers to an agent that is structurally similar to aldosterone, but differs slightly in composition, for example the replacement of one atom by an atom of a different element or functional group. For example, an analog of aldosterone is fludrocortisone that is 9-fluoro-11,17-dihydroxy-17-(2-hydroxyacetyl)-10,13-dimethyl-1,2,6,7,8,9,10,11,12,13,14,15,16,17-tetradecahydrocyclopenta[a]phenanthren-3-one.

In particular embodiment, the MR agonist according to the invention is selected from aldosterone, fludrocortisones, and deoxycorticosterone.

A further aspect of the invention relates to a combination of a glucocorticoid and a MR agonist for use in the treatment of an ocular inflammatory disease.

The combination of an MR agonist and a glucocorticoid potentiates the effect of the glucocorticoids and allows reducing the doses of glucocorticoids, thereby limiting their adverse side effects.

The glucocorticoids that may be used according to the invention include, but are not limited to, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisol one, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, anecortave acetate. and any of their derivatives,

The present also relates to a pharmaceutical composition (as herein after described) comprising an amount of at least one glucocorticoid and an amount of at least one MR agonist for use in the treatment of an ocular inflammatory disease.

The present invention also relates to a kit comprising at least one glucocorticoid and at least one MR agonist for use in the treatment of an ocular inflammatory disease.

The present invention also relates to an MR agonist for use in the prevention of the side effects induced by glucocorticoid during the treatment of an ocular inflammatory disease.

According to the invention, the active ingredients of the invention (e.g. MR agonists) are administered to the subject in a therapeutically effective amount.

By a “therapeutically effective amount” is meant a sufficient amount of the active ingredient to treat fluid accumulation in and/or under the retina at a reasonable benefit/risk ratio applicable to any medical treatment.

It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

As used herein, the term “subject” denotes a mammal, such as a rodent, a feline, a canine, and a primate. Preferably, a subject according to the invention is a human.

The active ingredients of the invention (e.g. MR agonists) may be combined with pharmaceutically acceptable excipients, and optionally sustained-release matrices, such as biodegradable polymers, to form pharmaceutical compositions.

The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The active ingredients of the invention shall be administered locally to the eyes of the subjected to be treated for avoiding the potential lethal effects of the administration of MR agonist in the systemic circulation.

Accordingly, the pharmaceutical composition of the invention is formulated for a local ocular route administration such as intravitreous, topical, periocular injections (sub conjunctival, peri bulbar, latero bulbar, retro bulbar, sub tenon, supra choroidal), intra or peri ocular implants (intra scleral, peri scleral, episcleral), intra vitreous implants or supra choroidal implants or particles or polymeric composition, or any releasing systems such as emulsions, solid non biodegradable or degradable implants or tablets, mini pumps or any topical formulations.

Preferably, the pharmaceutical compositions contain vehicles which are pharmaceutically acceptable for a formulation capable of being injected into the eye. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use in the eye include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria, virus and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The active ingredients of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions for the eyes are prepared by incorporating the active ingredients of the invention in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

Active ingredient may be also delivered directly to the eye by ocular tissue injection such as periocular, conjunctival, subtenon, intracameral, intravitreal, intraocular, subretinal, subconjunctival, retrobulbar, suprachoroidal or intracanalicular injections; by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or in the sclera (intrascleral) or supra choroidal or within the eye. Intracameral injection may be through the cornea into the anterior chamber to allow the agent to reach the trabecular meshwork. Intracanalicular injection may be into the venous collector channels draining Schlemm's canal or into Schlemm's canal.

For ophthalmic delivery, the active ingredient may be combined with ophthalmologically acceptable preservatives, co-solvents, surfactants, viscosity enhancers, penetration enhancers, buffers, sodium chloride, or water to form an aqueous, sterile ophthalmic suspension or solution. Solution formulations may be prepared by dissolving the active ingredient in a physiologically acceptable isotonic aqueous buffer. Further, the solution may include an acceptable surfactant to assist in dissolving the active ingredient. Viscosity building agents, such as hydroxymethyl cellulose, hydroxyethyl cellulose, methylcellulose, polyvinylpyrrolidone, or the like may be added to the compositions of the present invention to improve the retention of the compound.

In order to prepare a sterile ophthalmic ointment formulation, the active ingredient is combined with a preservative in an appropriate vehicle, such as mineral oil, liquid lanolin, or white petrolatum. Sterile ophthalmic gel formulations may be prepared by suspending the active ingredient in a hydrophilic base prepared from the combination of, for example, CARBOPOL®-940 (BF Goodrich, Charlotte, N.C.), or the like, according to methods known in the art. VISCOAT® (Alcon Laboratories, Inc., Fort Worth, Tex.) may be used for intraocular injection, for example. Other compositions of the present invention may contain penetration enhancing agents such as cremophor and TWEEN® 80 (polyoxyethylene sorbitan monolaureate, Sigma Aldrich, St. Louis, Mo.), in the event the active ingredient is less penetrating in the eye.

In a particular embodiment, the pharmaceutical composition of the invention is an ophthalmic drop formulation. The eye drop is provided in any formulation generally used, for example, in the form of an aqueous eye drop such as aqueous eye drop solution, aqueous eye drop suspension, viscous eye drop solution, solubilized eye drop solution and the like, or in the form of a non-aqueous eye drop such as a non-aqueous eye drop solution, non-aqueous eye drop suspension and the like. When the composition the present invention is prepared as an aqueous eye drop, it preferably contains an additive which is usually used in an aqueous eye drop. The examples of such an additive include preservatives, isotonic agents, buffering agents, stabilizer, pH regulators or the like.

In another particular embodiment, the active ingredients of the invention are delivered through a biodegradable ocular implant.

The implants can be formed in manner that the active ingredient is homogenously distributed or dispersed throughout the biodegradable polymer matrix. Additionally, the implants can be formed to release the active ingredient into an ocular region of the eye over various time periods. Thus, the active ingredient can be released from implants made according to the present invention for a period of time of, for example, 30-200 days.

The active ingredient can comprise from about 10% to about 90% by weight of the implant. In one variation, the agent is from about 40% to about 80% by weight of the implant. In a preferred variation, the agent comprises about 60% by weight of the implant

In a particular embodiment, the active ingredient can be homogeneously dispersed in the biodegradable polymer of the implant. The implant can be made, for example, by a sequential or double extrusion method. The selection of the biodegradable polymer used can vary with the desired release kinetics, patient tolerance, the nature of the disease to be treated, and the like. Polymer characteristics that are considered include, but are not limited to, the biocompatibility and biodegradability at the site of implantation, compatibility with the active ingredient of interest, and processing temperatures. The biodegradable polymer matrix usually comprises at least about 10, at least about 20, at least about 30, at least about 40, at least about 50, at least about 60, at least about 70, at least about 80, or at least about 90 weight percent of the implant. In one variation, the biodegradable polymer matrix comprises about 40% to 50% by weight of the implant.

Biodegradable polymers which can be used include, but are not limited to, polymers made of monomers such as organic esters or ethers, which when degraded result in physiologically acceptable degradation products. Anhydrides, amides, orthoesters, or the like, by themselves or in combination with other monomers, may also be used. The polymers are generally condensation polymers. The polymers can be crosslinked or non-crosslinked. If crosslinked, they are usually not more than lightly crosslinked, and are less than 5% crosslinked, usually less than 1% crosslinked. Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In certain variations, 25/75 PLGA and/or 50/50 PLGA copolymers are used. In other variations, PLGA copolymers are used in conjunction with polylactide polymers or polyurethanes.

Other agents may be employed in the formulation for a variety of purposes. For example, buffering agents and preservatives may be employed. Preservatives which may be used include, but are not limited to, sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. Examples of buffering agents that may be employed include, but are not limited to, sodium carbonate, sodium borate, sodium phosphate, sodium acetate, sodium bicarbonate, and the like, as approved by the FDA for the desired route of administration. Electrolytes such as sodium chloride and potassium chloride may also be included in the formulation.

The biodegradable ocular implants can also include additional hydrophilic or hydrophobic compounds that accelerate or retard release of the active ingredient. Additionally, release modulators such as those described in U.S. Pat. No. 5,869,079 can be included in the implants. The amount of release modulator employed will be dependent on the desired release profile, the activity of the modulator, and on the release profile of the active ingredient in the absence of modulator. Where the buffering agent or release enhancer or modulator is hydrophilic, it may also act as a release accelerator. Hydrophilic additives act to increase the release rates through faster dissolution of the material surrounding the drug particles, which increases the surface area of the drug exposed, thereby increasing the rate of drug diffusion. Similarly, a hydrophobic buffering agent or enhancer or modulator can dissolve more slowly, slowing the exposure of drug particles, and thereby slowing the rate of drug diffusion.

The release kinetics of the implants of the present invention can be dependent in part on the surface area of the implants. A larger surface area exposes more polymer and active ingredient to ocular fluid, causing faster erosion of the polymer matrix and dissolution of the active ingredient particles in the fluid. Therefore, the size and shape of the implant may also be used to control the rate of release, period of treatment, and active ingredient concentration at the site of implantation. At equal active ingredient loads, larger implants will deliver a proportionately larger dose, but depending on the surface to mass ratio, may possess a slower release rate. For implantation in an ocular region, the total weight of the implant preferably ranges, e.g., from about 200-15000 [mu]g, usually from about 1000-5000 [mu]g. In one variation, the total weight of the implant is about 1200 to about 1,800 [mu]g. In another variation, the total weight of the implant is about 2400 to about 3,600 [mu]g. Preferably, the implant has a weight between about 100 [mu]g and about 2 mg.

The implants of the invention are typically solid, and may be formed as particles, sheets, patches, plaques, films, discs, fibers, rods, and the like, or may be of any size or shape compatible with the selected site of implantation, as long as the implants have the desired release kinetics and deliver an amount of active ingredient that is therapeutic for the intended medical condition of the eye. The upper limit for the implant size will be determined by factors such as the desired release kinetics, toleration for the implant at the site of implantation, size limitations on insertion, and ease of handling. For example, the vitreous chamber is able to accommodate relatively large rod-shaped implants, generally having diameters of about 0.05 mm to 3 mm and a length of about 0.5 to about 10 mm. In one variation, the rods have diameters of about 0.1 mm to about 1 mm. In another variation, the rods have diameters of about 0.3 mm to about 0.75 mm. In yet a further variation, other implants having variable geometries but approximately similar volumes may also be used.

The biodegradable implants can be inserted into the eye by a variety of methods, including placement by forceps, by trocard, or by other types of applicators, after making an incision in the sclera. In some instances, a trocard or applicator may be used without creating an incision. In a preferred variation, a hand held applicator is used to insert one or more biodegradable implants into the eye. The hand held applicator typically comprises an 18-30 GA stainless steel needle, a lever, an actuator, and a plunger. Suitable devices for inserting an implant or implants into a posterior ocular region or site includes those disclosed in U.S. patent application Ser. No. 10/666,872.

The invention will be further illustrated by the following figures and examples. However, these examples and figures should not be interpreted in any way as limiting the scope of the present invention.

### FIGURES

FIG. 1 shows the severity of EAU after Intravitreal Injection of aldosterone and spironolactone (A) and after Intravitreal Injection of aldosterone and RU 26752.

FIG. 2 shows concentration of the different cytokines in the eye (i.e. MIP1α/CCL3, TNF-α, IFN-γ/IL-4, and IL-6) in treated and control animals.

### EXAMPLE

### Material & Methods

Animals:

Female Lewis rats (6- to 8-week old) weighing 230-250 g (Elevage Janvier, Le Genest Saint Isle, France) were used and handled in accordance with the ARVO Statement for the Use of Animals in Ophthalmic and Vision Research. Rats were anesthetized with intramuscular injection of ketamine (75 mg kg⁻¹; Virbac, Carros Cedex, France) and Largactil (0.5 mg kg⁻¹; Sanofi-aventis, Paris, France) before ocular injection and ET. At the end of the experiments, rats were anesthetized by intraperitoneal injection of pentobarbital (30 mg kg⁻¹; Sanofi-aventis) before blood collection by intracardiac puncture and then killed with a lethal dose of pentobarbital.

Induction and Scoring of Endotoxin-Induced Uveitis:

Endotoxin-induced uveitis was induced by footpad injection of 0.1 ml sterile pyrogen-free saline containing 500 μgkg⁻¹ LPS (from Salmonella typhimurium; Sigma-Aldrich). Animals were examined by slit lamp during the maximal severity of EIU developing in this model 24 h after the footpad injection. The intensity of clinical ocular inflammation was scored on a scale from 0 to 5 for each eye as described previously (Behar-Cohen F F, Parel J M, Pouliquen Y, Thillaye-Goldenberg B, Goureau O, Heydolph S et al. Iontophoresis of dexamethasone in the treatment of endotoxin-induced-uveitis in rats. Exp Eye Res 1997; 65: 533-545.): grade (0) indicates no inflammation; grade 1 indicates the presence of a minimal iris and conjunctival vasodilation but without the observation of flare or cells in the anterior chamber (AC); grade 2 indicates the presence of moderate iris and conjunctival vessel dilation but without evident flare or cells in the AC; grade 3 indicates the presence of intense iris vessels dilation, flare and less than 10 cells per slit lamp field in the AC; grade 4 indicates the presence of more severe clinical signs than grade 3, with more than 10 cells in the AC with or without the formation of a hypopyon; grade 5 indicates the presence of intense inflammatory reaction, fibrin formation in the AC and total seclusion of the pupil.

Clinical evaluation was performed in a masked manner by two observers. The mean clinical score of the two observers was recorded.

Intravitreal Injection Protocols:

Lewis rats were anesthetized by intraperitoneal injection of 0.15 mL pentobarbital (5.47 g/100 mL saline). Pupils were dilated by instillation of one drop of tropicamide 5%. One drop of tetracaine 1% was administered for local anesthesia. Intravitreal injections (10 μL) of aldosterone (20 nM and 200 nM) and spironolactone (10 μM) were performed in both eyes using sterile syringes fitted with a 30-gauge needle (Microfine; Becton Dickinson AG, Meylan, France). Intravitreal injections were performed simultaneously with EIU induction, as described.

Chemokine/Cytokine Multiplex Assay:

Aqueous humor and vitreous collected from each eye were pooled, diluted to obtain a final volume of 25 μL, and subjected to multiplex bead analysis. This method uses microspheres as the solid support for immunoassays (Vignali D A. Multiplexed particle-based flow cytometric assays. J Immunol Methods. 2000; 243:243-255.) and allows the titration of a greater number of cytokines with increased sensitivity than occurs with ELISA (Ooi K G, Galatowicz G, Towler H M, Lightman S L, Calder V L. Multiplex cytokine detection versus ELISA for aqueous humor: IL-5, IL-10, and IFNγ profiles in uveitis. Invest Ophthalmol Vis Sci. 2006; 47:272-277.). The rat cytokine/chemokine LINCO-16plex kit (Linco Research) was purchased from Invitrogen (Cergy-Pontoise, France). Chemokine MIP1α/CCL3, proinflammatory mediator TNF-α, cytokines IFN-γ/IL-4, and IL-6 were measured according to the manufacturer's instructions. The assay was performed in a 96-well filter plate with all the assay components provided in the kit. Standard curves for each cytokine were generated with a calibration kit (Bio-Rad, Hercules, Calif.). All incubation steps were performed at room temperature with medium orbital agitation and in the dark to protect the beads from light. Data acquisition and analysis were performed with the manager software version 4.1 (Bioplex; Bio-Rad) with four or five logistic parameters for standard curves. Detection levels for all the cytokines were 1 to 10 pg/mL.

Statistical Analysis:

Results are expressed as mean±SEM. The Mann-Whitney U test was used to determine differences between groups. P<0.05 was considered statistically significant.

Results

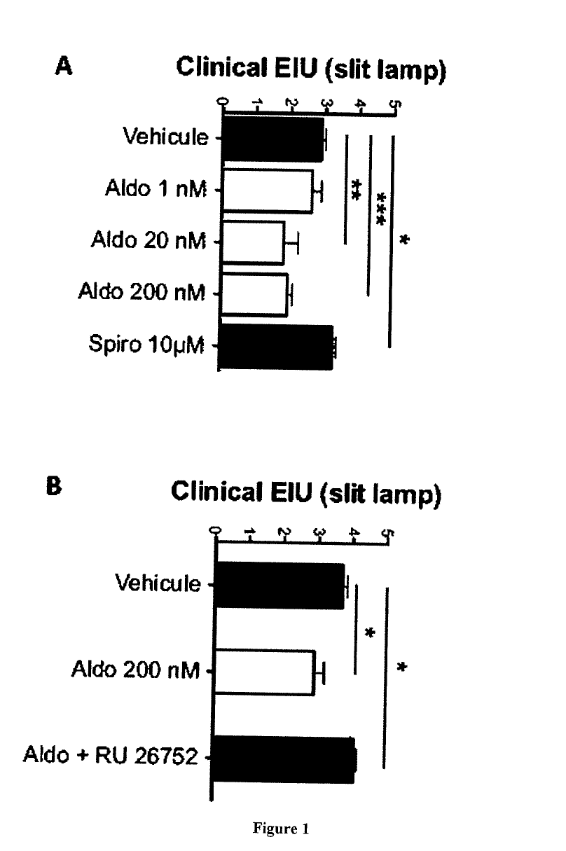

To evaluate the severity of uveitis, we examined rats at the slit lamp 24 hours after injection of LPS, therefore the peak of the disease. Clinical examination detected severity decrease statistically significant after aldosterone injection dose of 20 nM (p<0.05) and 200 nM (p<0.001). Injection of aldosterone 1 nM had no effect on the severity of uveitis (FIG. 1A). However intravitreal injection of spironolactone at a dose of 10 microM increased the intensity of uveitis in 24 hours (FIG. 1A). To check the effect of aldosterone was mediated by its binding to MR, we simultaneously injected aldosterone and a specific antagonist of MR (RU 26752). FIG. 1B shows that the combination of aldosterone with RU26752 exacerbates clinical severity. The anti-inflammatory effect of aldosterone is in part mediated by the MR.

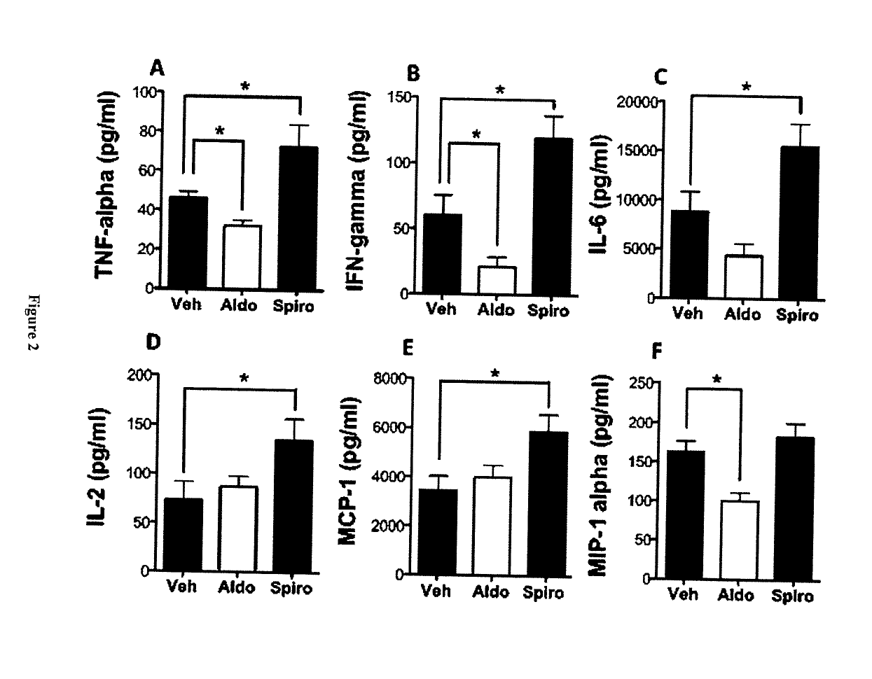

All tested cytokines and chemokines (i.e. MIP1α/CCL3, TNF-α, IFN-γ/IL-4, and IL-6) were significantly reduced in the ocular media of animals treated with aldosterone compared with control animals treated with the vehicle alone (FIG. 2). On the contrary, the tested cytokines and chemokines (i.e. MIP1α/CCL3, TNF-α, IFN-γ/IL-4, and IL-6) were significantly increased in the ocular media of animals treated with spironolactone (MR antagonist) compared with control animals treated with the vehicle alone (FIG. 2).

To evaluate the effect of intravitreal injection of 200 nM aldosterone and spironolactone 10 μM, immunohistochemical studies were performed on cryostat sections. An anti EDI antibody coupled with an Alexa488 secondary antibody was used to mark macrophages which are among the cells most involved in EIU. We conducted an EDI positive cell counts in histological sections 24 h after LPS injection and intravitreal injection of aldosterone or spironolactone. A decrease in the number of macrophages was observed after injection of aldosterone compared to eyes injected vehicle or spironolactone (p<0.05), confirming the anti-inflammatory effect already observed.

To evaluate the effect of aldosterone on resident microglial cells of the retina and ciliary body, we performed immunohistochemical studies with an anti-IBA-1 antibody coupled with a secondary antibody Alexa488 on cryostat sections 24 h after LPS injection and injection intravitreal aldosterone or spironolactone. After intra vitreous of aldosterone, we found that a greater number of microglial cells were then branched into a latent state (p<0.05) compared to eyes injected with the vehicle. After intravitreal injection of spironolactone, a larger number of microglial cells were round, and consequently active. The anti-inflammatory effect of aldosterone may be related to the deactivation of resident microglial cells.

Iris and ciliary body tissues are most involved in the EIU. In an attempt to understand the role of MR and GR in EIU model, we evaluated the expression of these two receptors in quantitative PCR in the iris/ciliary body 3 h, 6 h and 24 h after injection of LPS. We were surprised that the kinetics of MR and GR were not similar. 3 hours after induction of EIU (p<0.001), so before the infiltration of inflammatory cells, there was a decreased expression of MR, which was maintained up to 24 hours (p<0.001). On the contrary the decreased expression of GR was later in the 24th hour (p<0.01). Immunohistochemical studies confirmed the decrease in the rate of receptors in the iris. The decreased expression of MR and GR takes place mainly in the endothelial cells of vessels in the iris and iris stromal cells. At baseline, the binding of endogenous glucocorticoids to MR likely plays a role in anti-inflammatory. The reduction of early MR can be seen as a step prior to the onset of ocular inflammation.

Proteins present in eye fluids reflect the leaking of the blood-ocular barrier. At 6 hours, ie in the very early stages of the disease, aldosterone significantly reduced the rate of protein from the vehicle, whereas spironolactone had no effect confirming that aldosterone plays a role anti-inflammatory in the early stages of ocular inflammation.

The expression of MR and GR in the iris and ciliary body is increased after intravitreal injection of aldosterone (p<0.05). Intravitreal injection of spironolactone exacerbated the decrease in MR (p<0.05), already induced by LPS and has no effect on GR. Presumably, the anti-inflammatory effect of aldosterone in this model is partly due to the increased expression of MR.

### CONCLUSIONS

Altogether the results present the first in vivo evidence for the involvement of the mineralocorticoid receptor in the control of the ocular inflammation and demonstrated that activation of said receptor reduced the inflammation. Moreover, MR is shown to intervene in the control of the blood-aqueous barrier. MR agonists favour maintenance and/or restoration of ocular barriers in inflammation.

### REFERENCES

Throughout this application, various references describe the state of the art to which this invention pertains. The disclosures of these references are hereby incorporated by reference into the present disclosure.

1-9. (canceled)

10. A method of treating inflammation associated with an ocular inflammatory disease, comprising
administering to a subject exhibiting inflammation associated with an ocular inflammatory disease at least one mineralocorticoid receptor agonist in an amount effective to treat said inflammation, wherein said at least one mineralocorticoid receptor agonist includes aldosterone.

11. The method of claim 10, wherein said ocular inflammatory disease is selected from the group consisting of conjunctivitis, keratitis, endothelitis, uveitis, choroiditis, retinitis, retinochoroiditis, anterior uveitis, intermediate uveitis, posterior uveitis, pan uveitis and inflammatory optic neuropathies.

12. The method of claim 10, wherein said at least one mineralocorticoid receptor agonist is administered with a pharmaceutically acceptable carrier as a pharmaceutical composition.

13. The method of claim 10, wherein said at least one mineralocorticoid receptor agonist is administered via a local ocular route of administration selected from the group consisting of intravitreous, topical, periocular injections, intra or periocular implants, intra vitreous implants, supra choroidal implants, particles, polymeric compositions, emulsions, solid non biodegradable or degradable implants, and tablets, mini pumps and topical formulations.

14. The method of claim 13, wherein said periocular injection is subconjunctival, peribulbar, laterobulbar, retrobulbar, subtenon, suprachoroidal.

15. The method of claim 13, wherein said intra- or periocular implants are intrascleral, periscleral, or episcleral.
